(19) United States
(12) Patent Application Publication
(10) Pub. No.: US 2016/0135979 A1
(43) Pub. Date: May 19, 2016

(54) Neck Brace

(71) Applicant: Ronghua Lou, Jinhua (CN)

(72) Inventor: Ronghua Lou, Jinhua (CN)

(21) Appl. No.: 14/792,632

(22) Filed: Jul. 7, 2015

(30) Foreign Application Priority Data
Nov. 14, 2014 (CN) 201420679184.4

(51) Int. Cl. A61F 5/055

(52) U.S. Cl. CPC A61F 5/055

(57) ABSTRACT

The invention relates to a neck brace comprising a deformable brace, said deformable brace is provided with a removable cushion fixed thereon by a tie; said deformable brace is additionally provided with two movable sponge sleeves, and the two movable sponge sleeves slide along the deformable brace. the neck brace consists of a deformable brace, with one end supporting the head and the other end attached onto the body to distribute the weight of the head to other parts of the body through the brace, and reduce the pressure applied to cervical vertebra, thus protecting the cervical vertebra; a removable cushion is provided on one end of the brace, which can improve comfort degree of the product; two thickened sponge sleeves are provided in the middle of the structure, which can produce enhanced comfort when the brace is in contact with the body.

### CROSS-REFERENCE TO RELATED APPLICATIONS

This present application claims the benefit of Chinese Patent Application No. 201420679184.4 filed on Nov. 14, 2014, the contents of which are hereby incorporated by reference.

### TECHNICAL FIELD

The invention relates to a cervical protection device, particularly to a neck brace.

### BACKGROUND OF THE INVENTION

Whether in daily life or routine work, an effective cervical protection device is lacked. A neck guard is a neckerchief for protecting the neck. With the development of science and technology, the material of the neck guard is not limited to usual cloth anymore, and its function is not limited to keeping warm either. In terms of medical care, the neck guard is suitable for tepidity and physiotherapy against spondylopathy, postpartum problem, cervical strain, stomach cold, dysmenorrheal, cold fits, and other diseases.

The Application is the improvement of an existing patent disclosing a new sleep assist device with China Patent No. 201220700198.0, and the main features of the patent include “a tie and a square-shaped metal framework which bends forwards to longitudinally form a C-shaped section, said tie comprises two pieces, and one end of the said tie is provided with a hitching piece and said tie is hitched on the said metal framework through said hitching piece, said metal framework is externally wrapped with a buffer layer, and said hitching piece is hitched outside said buffer layer”. Its beneficial effects lie in that: it can be used for supporting the head when people take a nap in the seat with its simple comfortable and portable structure, specifically, people can put the device in front of the head, fix it on the back of the seat with the tie, and thus rest the forehead forward on a crossbeam of the upper segment of the metal framework, making it effectively support the head, adequately ensuring the comfort degree of nap, meanwhile, the design in which the metal framework is externally wrapped with a buffer layer makes it provide a more comfortable rest effect; it can be not only used normally for resting the forehead on the crossbeam of the upper segment, but also entirely put on the neck by bending the crossbeam of the upper segment downwards and reducing the opening degree of the C-shaped structure. In this way, people can hold and support the head by resting the chin on the upper segment of the metal framework. The sleep assist device has not only flexible and diverse service patterns but also safe and comfortable effects.

However, the above application and the neck guard products in the market can only provide very limited supporting for the neck, and such products as the backing block and the headrest can play a protective role only in a particular posture of the body; meanwhile, a neck protection ring for medical use can only distribute the weight of the head to the shoulders, and the head cannot move freely, and it is very inconvenient to use; furthermore, when in use, it suffers from various limitations such as those on body shape, posture and time and place for use. Therefore, in order to remedy the above defects, the prior art needs to be reasonably improved.

### SUMMARY OF THE INVENTION

Aiming at remedying the above defects, the invention provides a neck brace which can reduces the pressure applied to the cervical vertebra by distributing weight of the head to other parts of the body through the brace, thus protecting the cervical vertebra, and remedying the plural defects of the prior art.

In order to achieve the above objectives, the invention employs the following technical solution:

A neck brace comprises a deformable brace, which is provided with a removable cushion fixed thereon by a tie; said deformable brace is additionally provided with two movable sponge sleeves, and the two movable sponge sleeves slide along the deformable brace. The deformable brace has not only certain plasticity to deform and remain stationary under external force, but also certain strength to support when in use.

Accordingly, the device can have the following features that:

The main body of said deformable brace consists of metal filaments wrapped with an elastic material outside;

Said deformable brace comprises two ends according to the use patterns, wherein one end is attached in front of the lower abdomen of the body, and the other end supports the rear side of the head.

Said removable cushion can be mounted on any position of the deformable brace, and the initial positions of the two movable sponge sleeves are both kept in the middle of the deformable brace;

And said deformable brace can be an enclosed ring structure or an unclosed U-shaped structure.

Said neck brace of the invention has the following beneficial effects:

(1) The neck brace consists of a deformable brace, with one end supporting the head and the other end attached onto the body to distribute the weight of the head to other parts of the body through the brace, and to reduce the pressure applied to cervical vertebra, thus protecting the cervical vertebra;

(2) A removable cushion is provided on one end of the brace, which can improve comfort degree of the product;

(3) Two thickened sponge sleeves are provided in the middle of the structure, which can produce enhanced comfort when the brace is in contact with the body; and

(4) There is no limitation on body shape: the shape of the brace can be adjusted according to different statures of long, short, fat or thin, size of the head, length of the cervical vertebrae to fit individual conditions and provide optimal support for the neck;

(5) There is no limitation on posture: people can distribute the weight of the head through the brace and use the brace with different postures, whether standing, sitting, lying down, leaning backward or forward; and

(6) There is no limitation on time and place for use: because of its light and fashionable appearance, it can be used at home, in office and on transportation vehicles; it can reduce neck stress and keep a clear head when used in working hours; and it makes it easier to fall asleep when used in siesta; furthermore, it can make the body more relaxed while taking a rest or watching TV.

### BRIEF DESCRIPTION OF THE DRAWINGS

The invention will be further illustrated in details according to the drawings below.

FIG. 1 is a structural diagram of the neck brace according to an embodiment of the invention;

FIG. 2 is a schematic diagram of the brace cushion of the neck brace according to an Embodiment of the invention;

FIG. 3 is a reference diagram I of the use pattern of the invention;

FIG. 4 is a reference diagram II of the use pattern of the invention; and

FIG. 5 is a reference diagram III of the use pattern of the invention.

In the drawings:

1-Deformable brace; 2-Removable cushion; 3-Movable sponge sleeve; 4-Tie; 5-Zipper.

### DETAILED DESCRIPTION OF THE PREFERRED EMBODIMENT

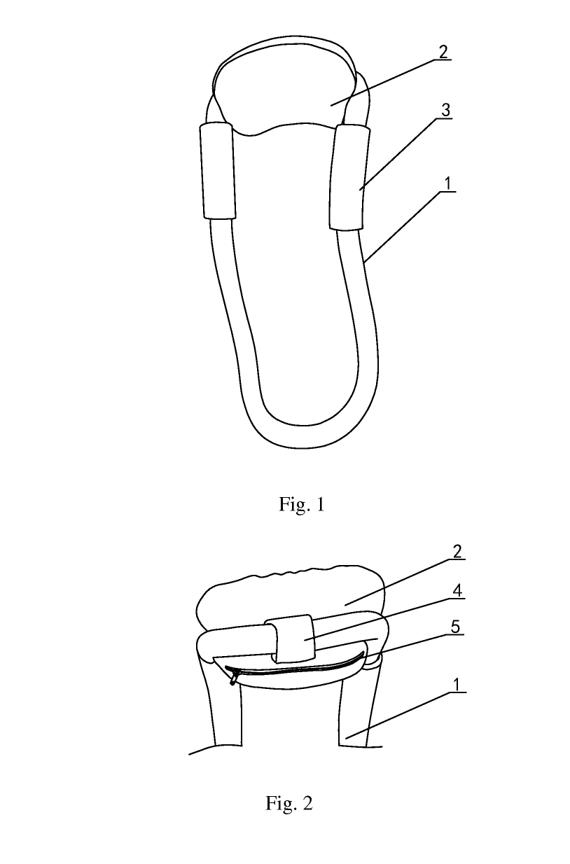

As shown in FIGS. 1-2, the neck brace according to an Embodiment of the invention comprises a deformable brace 1. Said deformable brace 1 is an irregular ring structure. And the deformable brace 1 is divided with two end structures according to the use patterns, wherein one end is attached onto the body, and the other end supports the head; the weight of the head can be distributed to other parts of the body through the brace, and the pressure applied to cervical vertebra is reduced, thus cervical vertebra is protected; and one end of the deformable brace 1 which supports the head is provided with a removable cushion 2 fixed thereon by a tie 4 to increase the comfort degree of the product;

Accordingly, said deformable brace 1 is additionally provided with two movable sponge sleeves 3 to produce enhanced comfort when the deformable brace 1 is in contact with the body, the initial positions of the two movable sponge sleeves 3 are both kept in the middle of the deformable brace 1, and the two movable sponge sleeves 3 can respectively slide along the deformable brace 1 to any position;

Furthermore, said removable cushion 2 is externally provided with a zipper 5 and the removable cushion 2 can be opened and closed by the zipper 5.

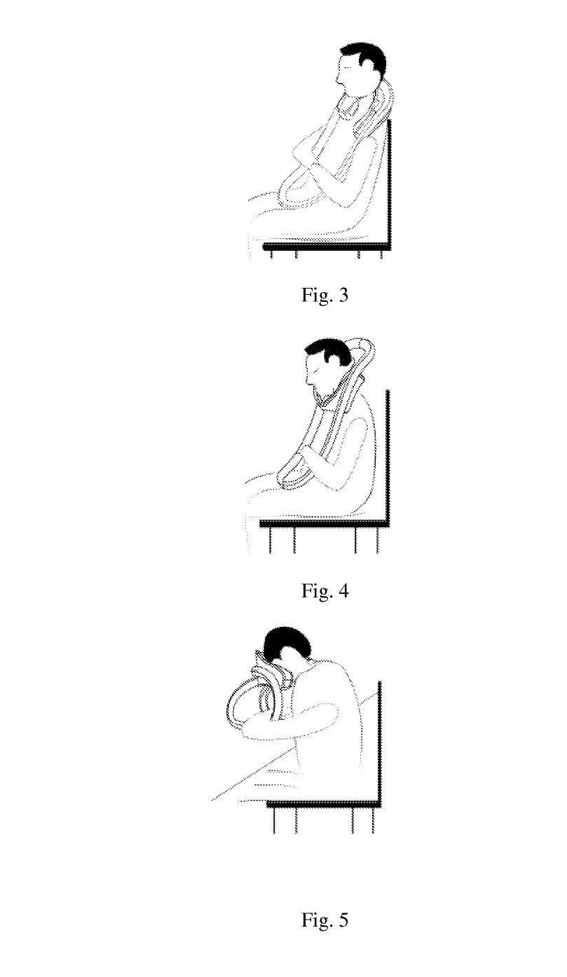

As shown in FIG. 3, when the body sits up straight or leans backward, the neck brace described above in the Embodiment of the Utility Model has one end underlaid behind the neck, and the other end placed in front of the body, a middle segment propped against shoulder blades, clavicles and the thoracic cavity, and a lower end attached to the hip, thus the head can well rest on the cushion of the brace, the weight of the head is shared by the neck, shoulders, chest and hip, and because the brace is attached to the body, the brace supports the head all along when the body posture is changed;

As shown in FIG. 4, when the body leans forward or the head is bowed, for example, writing or browsing on a mobile phone screen, the brace can be adjusted to narrow near the chin, the cushion is tightly attached to the chin and cheeks, the head is supported through the chin and cheeks, the lower part of the brace is attached to the abdomen and the hip, and the weight of the head is distributed; most often, a person is likely to bend forward to have a rest, for example, cat nap in the seat, then he can prop one end of the brace against the forehead, attach the middle segment of the brace to the abdomen, and prop the rear end of the brace against the hip and legs, or as shown in FIG. 5, one end of the brace is put on a table, and the brace can also achieve good effect of protecting cervical vertebra;

When in use, the brace can be selectively held with the hands or fixed on the waist by a tie according to the actual situation, and it is very convenient to use.

The above description of the Embodiments is to facilitate a person skilled in the art to understand and apply the technique of the Application, and it is obvious that a person skilled in the art can readily make various modifications to these embodiments, and apply the general principles illustrated herein to other embodiments without creative work. Therefore, the Application is not limited to above embodiments, and the improvements and modifications made by a person skilled in the art according to the disclosure of the Application shall fall within the protection scope of the Application.

What is claimed is:

1. A neck brace, comprising a deformable brace (1), wherein said deformable brace (1) has certain plasticity to deform and remain stationary under external force, and also has certain strength to support when in use; and said deformable brace (1) is provided with a removable cushion (2) thereon.

2. The neck brace according to claim 1, wherein said deformable brace (1) is additionally provided with two movable sponge sleeves (3), and the two movable sponge sleeves (3) slide along the deformable brace (1).

3. The neck brace according to claim 2, wherein said removable cushion (2) is mounted and fixed on the said deformable brace (1) by a tie (4).

4. The neck brace according to claim 3, wherein the main body of the said deformable brace (1) consists of metal filaments wrapped with an elastic material outside.

5. The neck brace according to claim 3, wherein said removable cushion (2) can be mounted on any position of the deformable brace (1).

6. The neck brace according to claim 3, wherein the initial positions of the two movable sponge sleeves (3) are both kept in the middle of the deformable brace (1).

7. The neck brace according to claims 1, wherein said deformable brace (1) can be an enclosed ring structure or an unclosed U-shaped structure.
